(19) United States
(12) Patent Application Publication
(10) Pub. No.: US 2020/0323689 A1
(43) Pub. Date: Oct. 15, 2020

(54) ADJUSTABLE WRAP-AROUND WEARABLE PILLOW

(71) Applicant: IDEABOY L.P., Santa Monica, CA (US)

(72) Inventors: HADI MORSHED, PACIFIC PALISADES, CA (US); ALEXANDER MORSHED, SANTA MONICA, CA (US); MASOUD NABAVI, TARZANA, CA (US)

(21) Appl. No.: 16/379,690

(22) Filed: Apr. 9, 2019

(51) Int. Cl. A61F 9/04; A47G 9/10; A42B 1/22; A42B 1/24; A61F 11/14

(52) U.S. Cl. CPC A61F 9/04; A47G 9/1081; A47G 9/1027; A42B 1/247; A61F 11/14; A42B 1/225

(57) ABSTRACT

An adjustable, wrap-around pillow includes a main, padded fabric section that is affixable to a user's head using fasteners. The pillow includes fabric pieces which are removably coupled to the main padded section that allow for the user's eyes and ears to be covered at the user's discretion. This gives the user the option of blocking light and/or sound, or conversely to enjoy audio or visual stimulation, while the pillow is being worn. The pillow includes support and cushioning surrounding the user's head and neck, and is usable is any sleeping or resting position, such as when lying down on a bed, couch, desk, floor, etc. or sitting up in a chair. When not in use, the pillow can be unfastened and folded flat for easy conveyance.

### CROSS-REFERENCE TO RELATED PATENT APPLICATION(S)

Not applicable.

### FIELD OF THE INVENTION

The present invention relates to the field of sleep accessories. More specifically, the present invention relates to the field of pillows for head and neck support.

### BACKGROUND OF THE INVENTION

In the field of sleep accessories, there are many types and styles of pillows available. While there are many conventional pillows of the type that enable a user to lay flat or their sides, there are also many pillows that can be used in either upright or prone positions. Examples of these can be found in U.S. Pat. No. 6,088,836, to de Cordova, and U.S. Pat. No. 6,651,256 to Swift. These prior art patents disclose sleep-enhancing devices that can be worn around a user's head, for example while traveling.

One problem with these existing wearable pillow designs is that they do not provide enough comfort or support for both the user's head and neck, and often also do not provide options for a user to see or hear while wearing such designs. Further, such designs often cause overheating for the user, making them unpleasant to wear for lengthy periods of time.

Both the prior art designs listed above are worn like helmets, and do not provide sufficient consideration for both a user's desire to see and to hear while the pillow is being worn. For example, the design of U.S. Pat. No. 6,088,836 does not provide openings for the user's eyes or ears. Similarly, the U.S. Pat. No. 6,651,256 does not allow for a user to hear at all, and its eye covering can be either worn or removed, but not removable so that it remains attached to the pillow to the allow the user to easily cover and uncover their eyes.

The design of U.S. Pat. No. 6,651,256 also covers the entire top of the user's head, meaning that the user's head is unable to “breathe” to keep the user cool. This design includes a neck roll which can be detachable and therefore also misplaced or lost. Also, this type of neck support still allows the user's head to tilt while being worn, similar to conventional neck rolls that are commonly worn by users on airplanes. Additionally, each of the two designs above are not easily foldable for storage when not in use.

Accordingly there is a need in the existing art for improvements to wearable pillow designs that provide adequate support for both the user's head and neck to promote satisfactory sleep while being worn in any sleeping position, and which are configured in one piece and to be one-size-fits-all. There is also a need in the existing art for wearable pillows that include attached, yet removable eye and ear coverings that still allow the user to easily cover and uncover eyes and ears and provide the option to see and hear, or not to see or hear, while wearing the pillow.

### BRIEF SUMMARY OF THE INVENTION

The present invention provides a wrap-around, wearable pillow that is adjustable and can be universally worn in any sleeping or resting position. The pillow includes a padded base section with fasteners that allow for the pillow to be removably applied about a user's face and head, so that the top of the head is exposed while still providing appropriate cushioning and support. The present invention also includes apertures or openings for the face and ears, and provides adjustable and closeable flaps for the eyes and ears, allowing the user to block out light and sound respectively, or alternatively to enable activities such as reading or the use of headphones while the pillow is being worn. The pillow also includes ample neck cushioning and support, and provides for a comfortable wearing and sleeping experience while traveling when it is applied to a user, so that a user can use on a trip or at home while in bed, instead of a conventional pillow.

It is one objective of the present invention to provide a comfortable pillow for home use or travel. It is another objective of the present invention to provide a pillow having appropriate head and neck cushion and support. It is another objective of the present invention to provide a pillow which promotes deep sleep and Rapid Eye Movement (REM) in the user.

There are many other specific objectives of the present invention. One such objective is to provide a pillow which includes the option of blocking light and/or sound to create a peaceful sleeping or resting environment. Still further, it is yet another objective of the present invention to provide a pillow which includes the option of leaving ears and/or eyes exposed for enjoying audio or visual entertainment while the head is comfortably encompassed in a pillow. Another objective of the present invention is to provide a symmetrical one-size-fits-all pillow due to a fastening mechanism that allows the present invention to have an adjustable size. Yet another objective is to provide a pillow having a flat design so that the pillow folds or collapses for easy transport. It is still another such objective of the present invention to provide a pillow having a zipper or other mechanism for inserting extra filling to adjust firmness, changing the filling, and washing the fabric thereof.

In one embodiment, the present invention is a wearable pillow, comprising a padded base section having both a head support portion and a neck support portion, and forming a wearable pillow with a plurality of openings when applied around a user's head and neck, a fastening mechanism, configured near at least one of opposing ends of the padded base section, for securely fastening the opposing ends to apply the padded base section to the user's head and neck, an eye mask configured to be applied to an eye opening among the plurality of openings in the wearable pillow to cover and uncover the user's eyes, the eye mask being attached to the padded base section at one end of the eye mask and removably attachable to the padded base section at a second end of the eye mask to block vision by the user, and a pair of ear cover pieces configured to be applied to ear openings among the plurality of openings in the wearable pillow to cover and uncover the user's ears, the pair of ear cover pieces being attached to the padded base section at one end of each cover piece and removably attachable to the padded base section at a second end of each ear cover piece to block sound by the user, wherein the padded base section and the head support portion are configured so that a top of the user's head remains uncovered, so that user's head keeps cool and air flows between the plurality of openings when the wearable pillow is applied.

In another embodiment, the present invention is a sleeping accessory, comprising a support cushion having a head support portion for protecting a head of a user, and a neck support portion for protecting a neck of the user, the support cushion having a padded base section forming both the head support portion and the neck support portion, and a plurality of removable coverings that are fixedly attached to the padded base section at position relative to the support cushion for each covering, and removably attached to the padded base section at opposing positions relative to the support cushion for each covering, the plurality of removable coverings including ear pieces and an eye mask, wherein the support cushion is configured to be worn around the head and the neck of the user by coupling opposing ends of the pillow together, so that a top of the user's head remains uncovered.

Other objects, embodiments, features, and advantages of the present invention will become apparent from the following description of the embodiments, which illustrate, by way of example, principles of the invention.

### BRIEF DESCRIPTION OF THE SEVERAL VIEWS OF THE DRAWINGS

The accompanying drawings, which are incorporated in and constitute a part of this specification, illustrate several embodiments of the invention and together with the description, serve to explain the principles of the invention.

FIG. 1 is a profile view of a wrap-around, wearable and adjustable pillow according to the present invention, being worn by a user with the peripheral components down;

FIG. 2 is a front facial view of the wrap-around, wearable and adjustable pillow according to the present invention, being worn by a user with the peripheral components up;

FIG. 3 is a rear perspective view of the wrap-around, wearable and adjustable pillow according to the present invention being worn by a user and showing the hook-and-loop fastener for affixing the wrap-around, wearable and adjustable pillow around the user's head;

FIG. 4 is a partial top view of the wrap-around, wearable and adjustable pillow while not in use, lying flat and able to be folded; and

FIG. 5 is another partial top view of the wrap-around, wearable and adjustable pillow while not in use, lying flat and able to be folded, on the opposing side to that shown in FIG. 4.

### DETAILED DESCRIPTION OF THE INVENTION

In the following description of the present invention, reference is made to the exemplary embodiments illustrating the principles of the present invention and how it is practiced. Other embodiments will be utilized to practice the present invention and structural and functional changes will be made thereto without departing from the scope of the present invention.

The present invention is, as noted above, a wrap-around, wearable and adjustable pillow 100. The pillow 100 is wearable by a user 120, and includes multiple coverings 140 for eyes 114 and ears 116 of the user 120 to enable visual and aural stimulation while the pillow 100 is being worn, and conversely to block out sight or sound. The pillow 100 is padded and provides ample support and cushion for the user's head 118 and neck 119, while at the same time remaining open at least at the top of the user's head 118 to prevent overheating while the pillow 100 is being worn.

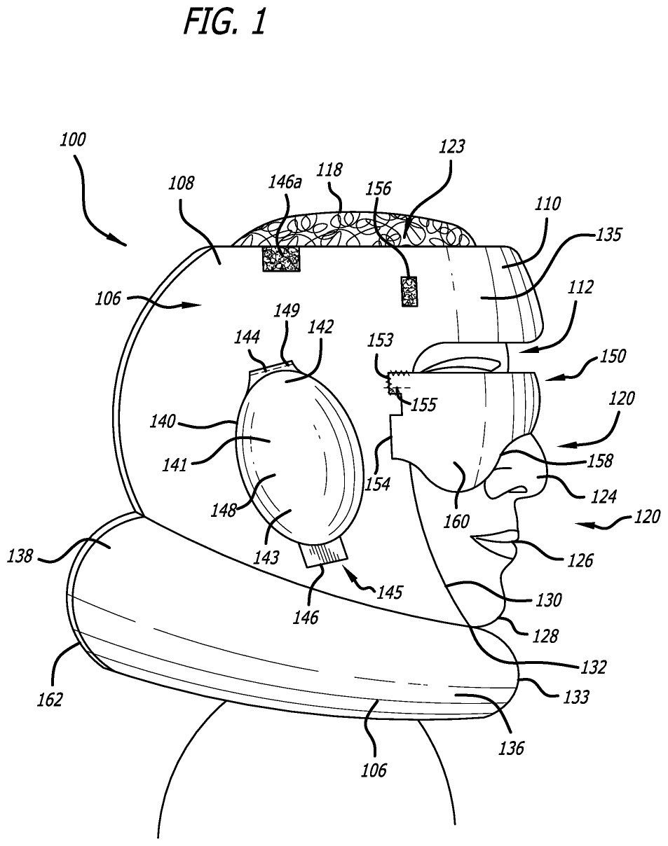

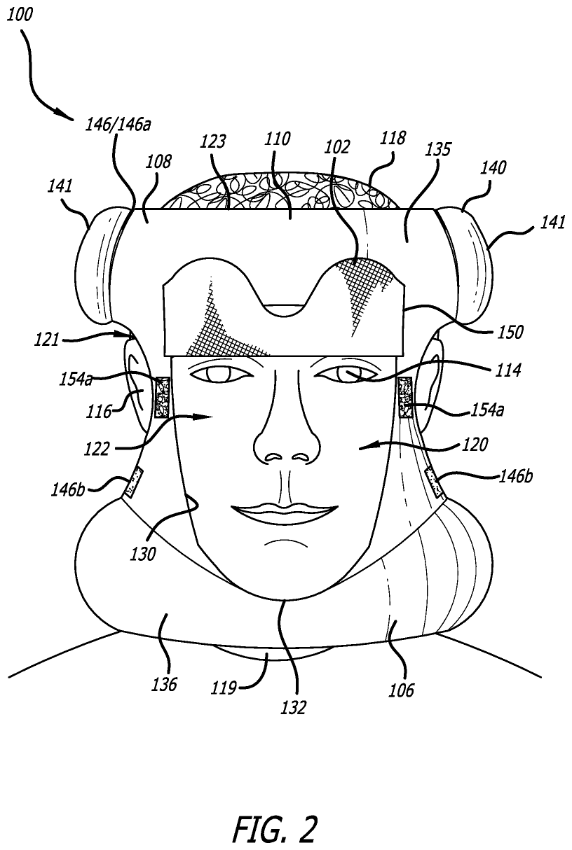

FIG. 1 is a profile view of a user 120 wearing the wrap-around, wearable and adjustable pillow 100 according to one exemplary embodiment of the present invention (with coverings 140 positioned down), and FIG. 2 is a front facial view of a user 120 wearing the wrap-around, wearable and adjustable pillow 100 according to this embodiment (with the coverings 140 positioned up). The pillow 100 includes an inner layer 102, which may be made for example of a soft fabric or cotton or silk-like material, and an outer layer 106 made of a more durable material, for example felt, denim, or neoprene. It is to be understood that the outer layer 106 of the padded base section 108 may also be made of any suitable material which allows a hook/male fastener 164 to adhere directly thereto. Such a material may be made from natural fibers such as wool, or synthetic fibers such as acrylic or neoprene. The outer layer 106 may therefore be constructed such that a fastener of a hook-style fastening mechanism 164 (i.e., male Velcro™) enables attach itself without the use of a supporting loop or pile fastener strip 162 (i.e., female Velcro™). Between the inner layer 102 and the outer layer 106 is a middle layer 104 which is made of a material providing cushion for the user 120, such as for example cotton, down, gel, Polyester fiber, Polystyrene beads, or foam. The inner layer 102, outer layer 106 and middle layer 104 together form a padded base section 108 which wraps around the user's head 118 and neck 119.

The padded base section 108 of the wearable pillow 100 includes a head support portion 135 and a neck support portion 136, which may be separated by stitching that partitions each of the head support portion 135 and the neck support portion 136 and allows them to be separately filled with differing amounts of padding material at the discretion of the user. This configuration allows for the user's head 118 and the user's neck 119 to be separately supported, and the user can customize the amount of padding in the wearable pillow 100 for each of the head support portion 135 and a neck support portion 136 by inserting or removing padding material via zipper(s) 166.

The pillow 100 also includes a plurality of coverings 140 that include ear flaps 141 and an eye mask 150. Each of the head support portion 135, the neck support portion 136 and plurality of coverings 140 may be comprised of a similar construct to that of the pillow 100 itself, in other words having an inner layer, a middle layer, and an outer layer. The ear flaps 141 may be coupled at one end 142 thereof to the padded base section 108 proximate to a position 144 where one opening 121 for the user's ears 116 is located on the padded base section 108, and removably attachable at the other end 143 to the padded base section 108 near an opposing side 145 to the position 144 relative to the opening 121, for each ear 116. In this manner, the user 120 can remove the ear flaps 141 while still having them attached to the padded base section 108 to hear activity around them or listen to audio, for example using headphones.

Similarly, the eye mask 150 may be coupled at one end 153 thereof to the padded base section 108 proximate to an upper position where an opening 122 for the user's eyes 114 is located on the padded base section 108, and removably attachable at sides thereof to the padded base section 108 via tabs 154 and using fastening mechanisms 154a when in a closed or covered configuration over the user's eyes 114. In this manner, the user 120 can remove the eye mask 150 while still having it attached (using a female tab or loop fastener 156, or directly to a surface of the outer layer 106) to the padded base section 108 to be able to see, for example to watch television while wearing the pillow 100. In this manner, the ear flaps 141 and eye mask 150 may be considered foldable over the user's ears 116 and eyes 114 respectively, while still remaining coupled to the pillow 100.

The eye mask 150 may be stitched to the pillow 100 using stitching 155, and as noted above affixed to outer layer 106 of the padded base section 108 with a fastener 156 such as a female or loop tab/fastener. The stitching 155, shown in FIG. 1 and FIG. 2 near the top end 153, allows the eye mask 150 to hinge upward, and in such a configuration the eye mask 150 may be coupled to one or more fastening mechanisms 156 on the outer layer 106 to keep the eyes 114 uncovered. Alternatively, the tabs 154 at sides of the eye mask 150 can also be folded to be attached to a surface of the outer layer 106 directly, at a position above the user's eyes 114, so that a user 120 can see. The eye mask 150 also includes padding 160 to effectively block out light when fully deployed, and may further have a contoured shape 158 to conform around the user's nose.

Similarly, ear flaps 141 are stitched to the pillow 100 using stitching 149, and affixed to outer layer 106 of the padded base section 108 with a fastener 146 such as hook fastener. The stitching 149 allows the ear flaps 141 to hinge upward, and the hook fastener 146 can be folded to be attached to outer layer 106 so that a user 120 can hear (as shown in FIG. 2). Fastening mechanisms 146a may also be provided, as shown in FIG. 1, on the outer layer 106 to which the fasteners 146 may be attached to keep the ear flaps 141 in an upward position to leave ears 116 uncovered. Similarly, fastening mechanisms 146b may also be provided on the outer layer 106 to which the fasteners 146 may be attached to keep the ear flaps 141 in a down position to cover ears 116. The ear flaps 141 also include padding 148 to block noise, and provide additional cushion and support if the user 120 is resting on his or her side.

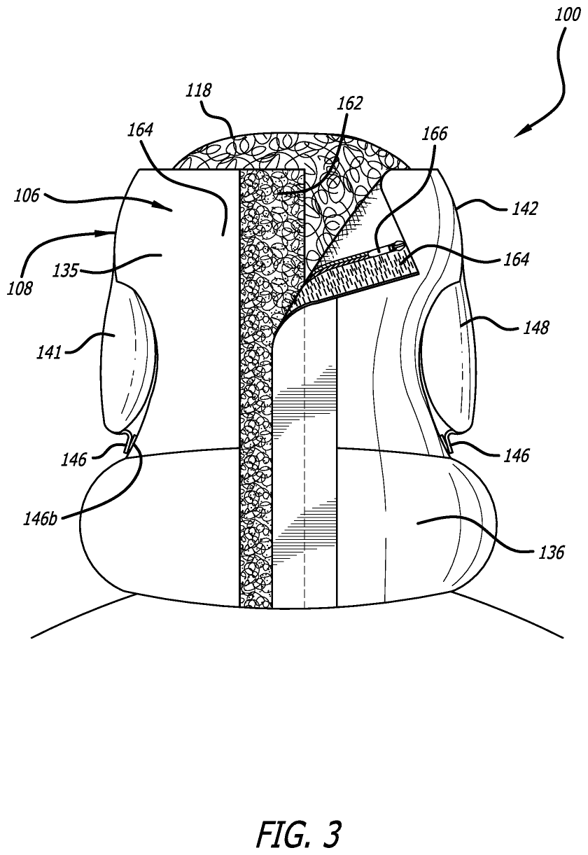

FIG. 3 is a rear perspective view of the wrap-around, wearable and adjustable pillow 100 being worn by a user 120, and showing fasteners 162 and 164 for affixing around the user's head 118. The pillow 100 is affixable, and sizeable, to the user's head 118 either by attaching hook fastener 164 to loop or pile fastener 162, or by attaching hook fastener 164 directly to the outer layer 106 of the padded base section 108. Once in place on the user's head 118, the pillow 100 may have multiple openings, such as an opening 123 at the top of the head 118, an opening 124 at the nose, an opening 112 at the forehead, an opening 126 at the mouth, an opening 128 at the chin, and an opening 130 for the face, all of which support air flow while the pillow 100 is being worn. Some or all of these openings may be provided, and it is to be understood that different embodiments may feature different ones of such openings, depending on product configuration and usage.

The pillow 100 includes additional padding, such as head padding 135 and neck support and padding 138 (in the neck portion 136), to support both the user's head 118 and the user's neck 119 in various positions whether the user 120 is lying down or sitting up. Forehead padding 110 may also be included to provide support for when the user 120 lies face down. The pillow 100 may also include a chin opening 132 for easy head maneuverability while providing a chin support area 133. The neck support and padding 138 covers the side and back of the neck 119, so that the user 120 can comfortably sleep or relax while lying down or sitting up. Zippers 166 may also be included alongside hook fastener 164 and loop fastener 162 (or anywhere else on the padded base section 108) so that a user 120 can remove material from the middle layer 104. Removing material from middle layer 104 may be done to more easily wash the inner layer 102 and outer layer 106, as well as to add or reduce padding according to the personal preference of the user 120.

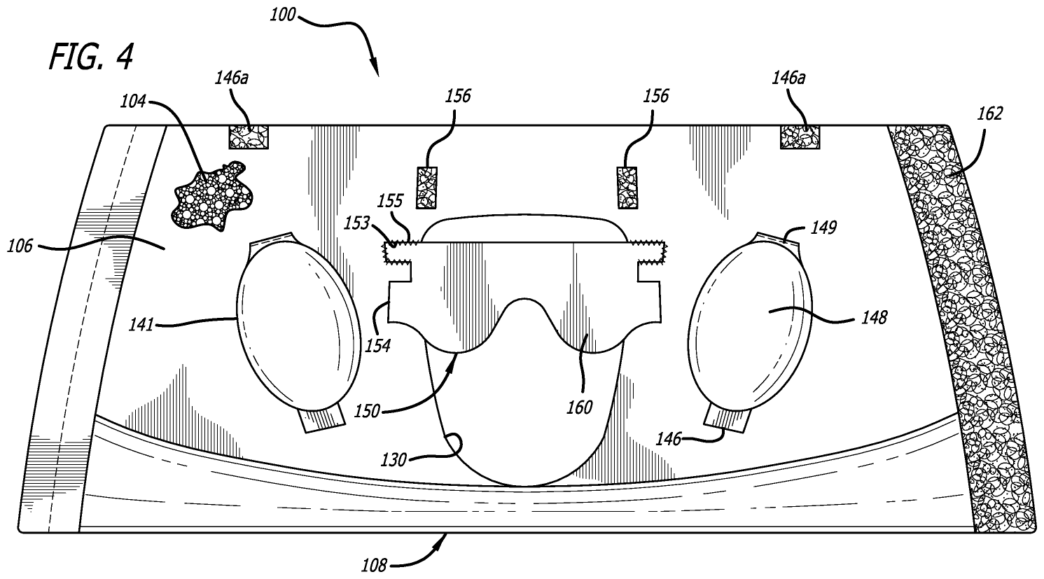

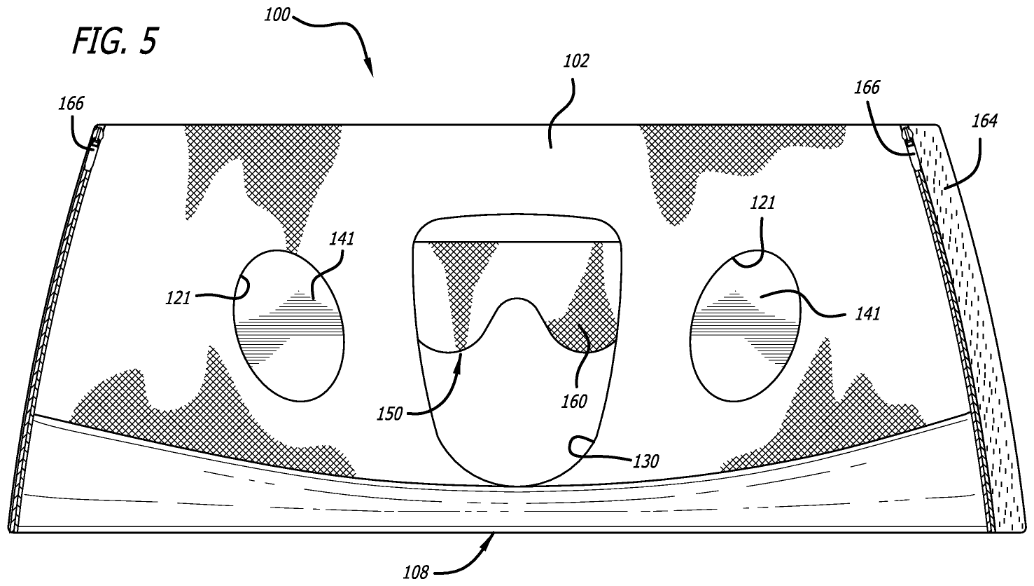

FIG. 4 and FIG. 5 show the wrap-around, wearable and adjustable pillow 100 while not in use, lying flat and able to be folded. As noted above, the pillow 100 is conveniently storable and transportable when not deployed around a user's head 118 and neck 119. The pillow 100 may folded, rolled, or otherwise maneuvered into a compact position for storage and travel. FIG. 4 is a top view of the pillow 100 showing the outer layer 106 face-up, and the coverings 140 (both ear flaps 141, and eye mask 150) as they would be in down or deployed positions. A portion of the outer layer 106 in FIG. 4 is shown fragmentary, to reveal the middle layer 104 and the material providing cushion for the user 120 therein.

FIG. 5 is a top view of the pillow 100 showing the inner layer 102 face up. As in FIG. 4, the coverings 140 are shown deployed over the ear openings 121 and eye opening 122.

The foregoing descriptions of embodiments of the present invention have been presented for the purposes of illustration and description. It is not intended to be exhaustive or to limit the invention to the precise forms disclosed. Accordingly, many alterations, modifications and variations are possible in light of the above teachings, may be made by those having ordinary skill in the art without departing from the spirit and scope of the invention. It is therefore intended that the scope of the invention be limited not by this detailed description. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

1. A wearable pillow, comprising:
a padded base section having both a head support portion and a neck support portion, and forming a wearable pillow with a plurality of openings when applied around a user's head and neck;
a fastening mechanism, configured near at least one of opposing ends of the padded base section, for securely fastening the opposing ends to apply the padded base section to the user's head and neck;
an eye mask configured to be applied to an eye opening among the plurality of openings in the wearable pillow to cover and uncover the user's eyes, the eye mask being attached to the padded base section at one end of the eye mask and removably attachable to the padded base section at a side of the eye mask to block vision by the user when the eye mask is deployed over the user's eyes; and
a pair of ear cover pieces configured to be applied to ear openings among the plurality of openings in the wearable pillow to cover and uncover the user's ears, the pair of ear cover pieces being attached to the padded base section at one end of each cover piece and removably attachable to the padded base section at a second end of each ear cover piece to block sound by the user when the pair of ear cover pieces are deployed over the user's ears,
wherein the padded base section and the head support portion are configured so that a top of the user's head remains uncovered, so that user's head keeps cool and air flows between the plurality of openings when the wearable pillow is applied.

2. The wearable pillow of claim 1, wherein the fastening mechanism is a hook fastener, the hook fastener being applicable directly to a surface of the padded base section to removably adhere the opposing ends together.

3. The wearable pillow of claim 1, wherein the fastening mechanism includes a hook fastener configured at one opposing end of the padded base section and a loop fastener configured at a second end of the padded base section, the hook fastener being applicable to the loop fastener to removably adhere the opposing ends together.

4. The wearable pillow of claim 1, further comprising at least one zipper in the padded base section, the at least one zipper enabling addition and removal of a cushioning material to the padded base section, and washing of the wearable pillow.

5. The wearable pillow of claim 1, wherein the eye mask is removably attachable to the padded base section with a fastening mechanism at the second end of the eye mask, the fastening mechanism comprising either a hook fastener applicable directly to the padded base section, or a hook fastener applicable to a loop fastener on the padded base section.

6. The wearable pillow of claim 1, wherein the eye mask is coupled to the padded base section at the first end by a stitching, so as to be permanently coupled to the padded base section at the first end.

7. The wearable pillow of claim 1, wherein the eye mask is coupled to the padded base section at the first end by a fastening mechanism so as to be entirely removably coupled to the padded base section, the fastening mechanism comprising either a hook fastener applicable directly to the padded base section at the first end, or a hook fastener applicable to a loop fastener on the padded base section at the first end.

8. The wearable pillow of claim 1, wherein the pair of ear cover pieces are each removably attachable to the padded base section with a fastening mechanism at the second end of each ear cover piece, the fastening mechanism comprising either a hook fastener applicable directly to the padded base section, or a hook fastener applicable to a loop fastener on the padded base section.

9. The wearable pillow of claim 1, wherein the pair of ear cover pieces are each coupled to the padded base section at first ends of each ear cover piece by a stitching so as to be permanently coupled to the padded base section at the first ends.

10. The wearable pillow of claim 1, wherein the pair of ear cover pieces are each coupled to the padded base section at first ends of each ear cover piece by a fastening mechanism so as to be entirely removably coupled to the padded base section, the fastening mechanism comprising either a hook fastener applicable directly to the padded base section at the first ends, or a hook fastener applicable to a loop fastener on the padded base section at the first ends.

11. A sleeping accessory, comprising:
a support cushion having a head support portion for protecting a head of a user, and a neck support portion for protecting a neck of the user, the support cushion having a padded base section forming both the head support portion and the neck support portion, the padded base section being partitioned between the head support portion and the neck support portion so that different amounts of a padded material are separately positioned in each of the head support portion and the neck support portion; and
a plurality of removable coverings that are fixedly attached to the padded base section at position relative to the support cushion for each covering, and removably attached to the padded base section at opposing positions relative to the support cushion for each covering, the plurality of removable coverings including ear pieces and an eye mask,
wherein the support cushion is configured to be worn around the head and the neck of the user by coupling opposing ends of the pillow together, so that a top of the user's head remains uncovered.

12. The sleeping accessory of claim 11, wherein the plurality of removable coverings are configured to allow the user to cover a user's ears and a user's eyes to block out sound and light, or to uncover the ears and the eyes to enable hearing and enable vision.

13. The sleeping accessory of claim 11, wherein the opposing ends are coupled together using a fastener.

14. The sleeping accessory of claim 13, wherein the fastener is a hook fastener, the hook fastener being applicable directly to a surface of the padded base section to removably adhere the opposing ends together.

15. The sleeping accessory of claim 13, wherein the fastener includes a hook component configured at one opposing end of the padded base section and a loop component configured at a second end of the padded base section, the hook component being applicable to the loop component to removably adhere the opposing ends together.

16. The sleeping accessory of claim 11, further comprising at least one zipper in the support cushion, the at least one zipper enabling addition and removal of a cushioning material to the support cushion.

17. The sleeping accessory of claim 12, wherein the eye mask is removably attachable to the support cushion with a fastener at a side of the eye mask, the fastener comprising either a hook fastener applicable directly to the support cushion, or a hook fastener applicable to a loop fastener on the support cushion.

18. The sleeping accessory of claim 17, wherein the eye mask is coupled to the support cushion at a first end by a stitching, so as to be permanently coupled to the support cushion at the first end.

19. The sleeping accessory of claim 17, wherein the eye mask is coupled to the support cushion at the first end by a fastening mechanism so as to be entirely removably coupled to the support cushion, the fastening mechanism comprising either a hook fastener applicable directly to the support cushion at the first end, or a hook fastener applicable to a loop fastener on the support cushion at the first end.

20. The sleeping accessory of claim 12, wherein the ear pieces are each removably attachable to the support cushion with a fastener at a second end of each ear piece, the fastener comprising either a hook fastener applicable directly to the support cushion, or a hook fastener applicable to a loop fastener on the support cushion.

21. The sleeping accessory of claim 20, wherein the ear pieces are each coupled to the support cushion at first ends of each ear piece by a stitching so as to be permanently coupled to the support cushion at the first ends.

22. The sleeping accessory of claim 20, wherein the ear pieces are each coupled to the support cushion at first ends of each ear piece by a fastener so as to be entirely removably coupled to the support cushion, the fastener comprising either a hook fastener applicable directly to the support cushion at the first ends, or a hook fastener applicable to a loop fastener on the support cushion at the first ends.
